SEQUENCE LISTING (4 sequences)

SEQ ID NO: 1 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 2 - DNA, 2061 bp, Bos taurus
atcttttctatcaaccccaaaactttggcacaatgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgagaagtcacttcacactctctttggagatgaattgtgtaaagttgcatcccttcgtgaaacctatggtgacatggctgactgctgtgagaaacaagaacctgagagaaatgaatgcttcttgtcacacaaagatgatagccctgatctacctaaactcaaacctgaccccaatactttgtgtgacgagtttaaggccgatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagggaaaaggtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtgcacaaggaatgctgccatggagacctacttgaatgcgcagatgacagggcggaccttgccaagtacatatgtgataatcaagatacaatctccagtaaactgaaggaatgctgtgataagcctttgttggaaaaatcccactgcattgctgaggtagaaaaagatgccatacctgaaaacttgcccccattaactgctgactttgctgaagataaggatgtatgcaaaaactatcaagaagcaaaggatgccttcctgggctcatttctttatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaattaaacaaaactgtgaccaattcgaaaaacttggagagtatggattccaaaatgcgctcatagttcgttacaccaggaaagtaccccaagtgtcaactccaactctcgtggaggtttcaagaagcctaggaaaagtgggtactaggtgttgtacaaaaccggaatcagaaagaatgccctgtacagaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtagacaagtgctgcgcagctgatgacaaagaagcctgctttgctgtggagggtccaaaacttgttgtttcaactcaaacagccttagcctaaacacgacacacactcaaggcatctcagcctacctgagagtaagagaaaagagaatgaaactagagcttatcatctgttttcttttctgttgagtagttaaaccaacactctcactactaaagaataaactttaatatttttgcgcttcttttgttgttgctacaattaataaaaaatgaaaagatctaaaaaaaaaaaaaaaaaa

SEQ ID NO: 3 - protein, 15 aa, Bos taurus
CDEFKADEKKFWGKY

SEQ ID NO: 4 - protein, 15 aa, Bos taurus
DSPDLPKLKPDPNTL

(19) United States
(12) Patent Application Publication
(10) Pub. No.: US 2014/0294850 A1
(43) Pub. Date: Oct. 2, 2014

(54) EARLY CHILDHOOD MEMBRANOUS NEPHROPATHY DUE TO CATIONIC BOVINE SERUM ALBUMIN

(71) Applicants: Pierre Ronco, Paris (FR); Hanna Debiec, Paris (FR)

(72) Inventors: Pierre Ronco, Paris (FR); Hanna Debiec, Paris (FR)

(73) Assignee: Institut National de la Sante et de la Recherche Medicate (INSERM), Paris (FR)

(21) Appl. No.: 14/237,907

(22) PCT Filed: Sep. 1, 2011
(86) PCT No.: PCT/EP2011/065112

§ 371 Date: Jun. 17, 2014

(30) Foreign Application Priority Data
Aug. 10, 2011 (IB) PCTIB2011/ 002083

(51) Int. Cl. G01N 33/53; C07K 16/18

(52) U.S. Cl. CPC G01N 33/53; C07K 16/18
USPC 424/140.1; 530/387.9; 436/501; 435/7.92; 435/7.1; 426/580; 426/587; 426/588

(57) ABSTRACT

The invention concerns a biomarker for diagnosing or prognosing childhood Membranous Nephropathy (MN), said biomarker is (i) cationic Bovine Serum Albumin (BSA), and/or (ii) an antibody that binds to a polypeptide of sequence SEQ ID NO: 3. The invention further concerns an antibody or antibody fragment or a composition comprising such an antibody or antibody fragment, wherein said antibody or antibody fragment is specific to an amino acid sequence SEQ ID NO: 3. The invention also concerns a foodstuff likely to contain BSA or cow milk or cow milk extracts, wherein said foodstuff is depleted in BSA.

### FIELD OF THE INVENTION

The invention concerns a biomarker for diagnosing or prognosing of childhood Membranous Nephropathy (MN), said biomarker is (i) cationic Bovine Serum Albumin (BSA), and/or (ii) an antibody that binds to a polypeptide of sequence SEQ ID NO: 3. The invention further concerns an antibody or antibody fragment or a composition comprising such an antibody or antibody fragment, wherein said antibody or antibody fragment is specific to an amino acid sequence SEQ ID NO: 3. The invention also concerns a foodstuff likely to contain BSA or cow milk or cow milk extracts wherein said foodstuff is depleted in BSA.

### BACKGROUND OF THE INVENTION

Membranous Nephropathy (MN) is the most common cause of nephrotic syndrome in adults, but is rare in children. The central pathogenesis involves the formation of subepithelial immune deposits which are responsible for functional impairment of the glomerular capillary wall. Two major antigens have been recently identified. The first is neutral endopeptidase (NEP), the alloantigen involved in neonatal cases of membranous nephropathy, and the second is the M type phospholipase A2 receptor (PLA2R), identified in idiopathic membranous nephropathy. Idiopathic membranous nephropathy is considered an autoimmune disease, while secondary forms involve exogenous antigens such as viral, bacterial, and tumoral antigens. It is likely that a growing number of “idiopathic” membranous nephropathy will be reclassified as “secondary” once non-glomerular antigens are identified.

Epidemiological surveys have identified nutritional elements as risk factors for development of autoimmunity in genetically susceptible persons. Bovine serum albumin (BSA) is one of the cow's milk and beef proteins that can escape from the intestinal barrier and thus induces formation of anti-BSA antibodies. In modern dietary culture, food ingredients are subjected to a variety of processing conditions that may induce modification of food proteins, which could change their digestion and allow their passage into the blood stream.

### SUMMARY OF THE INVENTION

The inventors now report a mechanism for childhood membranous nephropathy involving anti-BSA antibodies and a modified food derived antigen. Indeed, the inventors identified, in the full length BSA amino acid sequence (herein after as SEQ ID NO: 1), an epitope found in the amino acid sequence SEQ ID NO: 3. This epitope is detected in biological samples such as blood or kidney sample of Membranous Nephropathy (MN) patients preferably in early childhood MN patients. This epitope is notably in the form of cationic BSA that appears to become planted in the anionic glomerular capillary wall, thus inducing in situ formation of immune complexes.

Thus, in a first aspect, the invention concerns, a method for diagnosing or prognosing MN in a patient, preferably a child, which method comprises detection of (i) cationic Bovine Serum Albumin (BSA) or of a cationic polypeptide comprising sequence SEQ ID NO: 3 and/or (ii) an antibody that binds to a polypeptide of sequence SEQ ID NO: 3, in a biological sample from said child patient. Preferably, said cationic BSA or said cationic polypeptide has an isoelectric point (pI) above 7, preferably above 7.5 or 8, more preferably above 9, even more preferably between 9.5 and 10.0. Preferably, said polypeptide may contain a sequence or having a sequence having at least 85%, or at least 88%, or at least 90% identity to SEQ ID NO: 1 more preferably, of sequence SEQ ID NO: 3.

Preferably, said antibody may be IgG1 and/or IgG4.

In some embodiments, said biological sample may be a fluid sample or one or more cells, or a tissue sample from a patient; preferably a serum or plasma sample. Preferably, said biological sample may be a tissue sample from a patient, preferably a renal sample. Fluid samples may be urine, whole blood, plasma, tissue exudates and secretions. Suitable samples include all samples that may contain, naturally or pathologically, antibodies or circulating proteins or protein deposits such as cationic BSA or the polypeptide of sequence SEQ ID NO: 1 or a fragment thereof, or polypeptide comprising the sequence SEQ ID NO: 3.

In another aspect, the invention concerns the use of (i) cationic Bovine Serum Albumin (BSA) and/or (ii) a cationic polypeptide comprising sequence SEQ ID NO: 3, preferably, a cationic polypeptide comprising sequence SEQ ID NO: 1 or at least a fragment thereof and/or (iii) an antibody that binds to a polypeptide of sequence SEQ ID NO: 3 as a biomarker of Membranous Nephropathy (MN), preferably, childhood MN. Typically, said cationic BSA or cationic polypeptide has an isoelectric point (pI) above 7, preferably above 7.5 or 8, more preferably above 9, even more preferably between 9.5 and 10.0

In another aspect of the invention, the invention concerns an isolated antibody or antibody fragment which binds specifically to an amino acid sequence SEQ ID NO: 3. Said antibody recognizes specifically BSA but not Human serum albumin or Pig serum albumin or Goat serum albumin. Moreover said antibody binds specifically to sequence SEQ ID NO: 3 but not to adjacent sequences such as SEQ ID NO: 4.

The invention equally concerns a composition comprising such an antibody or antibody fragment.

The invention provides a kit for detecting cationic BSA or cationic polypeptide comprises sequence SEQ ID NO: 3 in a biological sample of an individual comprising:
a) means for capturing circulating BSA
b) an antibody or antibody fragment which binds specifically to an amino acid sequence SEQ ID NO: 3 or a composition comprising such an antibody or antibody fragment and/or
c) means for determining the isoelectric point (pI) of a polypeptide, preferably said pI measuring means may be an isoelectro-focusing gel, discontinuous electrophoresis gels, a ion-exchange chromatography column or a CM-Sephadex column.

Said means for capturing circulating BSA may be a binding partner of cationic BSA or a molecule having affinity for cationic molecules or any purification system. Preferably, said binding partner of cationic BSA may be said antibody or antibody fragment as defined in b).

Typically, said antibody or an antibody fragment is coupled to beads and/or to a detecting mean. Typically, said beads are, agarose beads. Said detecting means may be an enzyme labeled with a fluorescent compound or metal or a chemiluminescent compound.

The invention also provides an in vitro method for diagnosing or prognosing MN in a child patient comprises the steps of (i) purifying BSA or polypeptide comprises sequence SEQ ID NO: 3 from a biological sample, preferably by an affinity chromatography such as by using immobilized anti-BSA antibody or antibody fragment; (ii) determining if the isoelectric point of said BSA or of said polypeptide is above 7; and (iii) deducing therefrom that the individual is affected by MN.

Typically, said immunopurified BSA or said immunopurified polypeptide from patient serum is separated on nondenaturing isoelectric focusing gel (pH3-10) and transferred into PVDF or other membrane. The separated proteins are detected with specific anti-BSA antibody, or preferably antibodies specific for peptide SEQ ID NO: 3 labeled by covalently linking to an enzyme labeled with a fluorescent compound or metal or label with a chemiluminescent compound (FIG. 3).

According to another aspect, the invention provides a kit for detecting antibodies directed against BSA, in a biological sample of an individual comprising:

(i) a polypeptide comprising sequence SEQ ID NO: 3; and/or,

(ii) a polypeptide comprising sequence SEQ ID NO: 1 and/or,

(iii) an homologous polypeptide comprising or consisting of a sequence having at least 90% identity with sequence SEQ ID NO: 1; and/or,

(iv) a fragment of said polypeptide defined in (ii) and/or fragment of said homologous polypeptide sequence defined in (iii) provided said fragment or homologous polypeptide binds an antibody which binds specifically to an amino acid sequence SEQ ID NO: 3, and optionally,

(v) an antibody or antibody fragment which binds specifically to an amino acid sequence SEQ ID NO: 3 or a composition comprising such an antibody or antibody fragment and/or,

(vi) a polypeptide of sequence SEQ ID NO: 4.

Detection of specific anti-BSA antibodies is provided herein by a method comprising the steps of i) determining the presence of antibodies which binds specifically to an amino acid sequence SEQ ID NO: 1, and ii) determining the level of antibodies which bind specifically to an amino acid sequence SEQ ID NO: 3 in a serum sample from children having nephrotic syndrome. The antibodies can be detected by an immunoassay wherein an antibody-antigen complex is formed.

In one embodiment, the serum albumin has a bovine, a human, a goat or pig origin. The proteins are deposited or immobilized on a solid support. The antigens used are native highly pure, isolated by affinity chromatography. The support can be in the format of a dipstick, a test strip, a latex bead, a microsphere or a multi-well plate. The device further comprises a detection antibody which is specifically reactive with human IgG and labeled by covalently linking to an enzyme label compound. (FIG. 4)

According to said method, a polypeptide of sequence SEQ ID NO: 3 and a polypeptide of sequence SEQ ID NO: 4 (aa132-146) from SEQ ID NO:1 are used. The polypeptide of sequence SEQ ID NO: 4 is a negative control. The peptide solution is covalently immobilized in the multi well plate (ELISA). In this ELISA a known amounts of antigens (the peptides with a spacer sequence GSGS, a biotin residue at the N-terminus and a carboxamide at the C-terminus) are covalently immobilized in the well of NeutrAvidin coated plates, and then, the sample, e.g. serum or plasma, suspected of containing antibodies to BSA, is washed over the surface so that the antibodies can bind to the immobilized peptides. The surface is washed to remove any unbound protein and a detection antibody is applied to the surface. The detection antibody is specifically reactive to the subject and labeled by covalently linking to an enzyme label with a fluorescent compound or metal or label with a chemiluminescent compound (FIG. 5).

As positive control antibodies against BSA, preferentially antibodies specific for peptide SEQ ID NO: 3 can be used. Antibodies can be raised against the peptide SEQ ID NO: 3 by one of skill in the art. The process of immunization to elicit antibody production in a mammal or the generation of hybridomas to produce monoclonal antibodies, and the purification of antibodies may be performed by described in “Current Protocols in Immunology and Antibodies”. The immunoaffinity purified antibodies will be further labeled with tag component according to the actual needs.

The invention further provides a method for treating a child with MN, said method comprising removing antibodies which bind to a polypeptide of sequence SEQ ID NO: 1, preferably antibodies which bind specifically to a polypeptide of sequence SEQ ID NO: 3, from a subject.

Said method comprising the immunoabsorption of specific antibodies against BSA helps reduce the amount of circulating antibodies, thereby reducing in situ formation of immune complexes with planted cationic BSA in the glomerular capillary wall. Immunoabsorption of antibodies against BSA can occur by passing the blood, serum or plasma over immobilized BSA. Recombinant or native bovine serum albumin can be immobilized on inert and sterile matrices that are known in the art, such as sepharose. The native bovine albumin is purified from bovine serum. Methods of native protein purification are well known to one skilled in the art. The antibodies against BSA will bind to the immobilized BSA and remain bound to the matrix indirectly. The blood, serum or plasma is then collected. The resultant blood, serum, or plasma should have no detectable or reduced anti-BSA antibodies. The immunoabsorption procedure should be conducted under sterile conditions. The collected blood, serum or plasma that is now depleted of anti-BSA antibodies can be transfused back into the patient.

According to another aspect, the invention provides a foodstuff likely to contain cow milk or cow milk extracts, wherein said foodstuff is depleted in BSA.

Preferably, said foodstuff may be a dairy food.

Typically, said foodstuff contains powdered milk, condensed milk, including skimmed products as well as unskimmed ones.

Preferably, said foodstuff may be a child food.

Typically, said foodstuff may contain less than 1%, 0.5%; 0.2%, 0.02%; 0.01% of BSA.

According to another aspect, the invention provides a method of preventing or treating a MN in a patient, which method comprises administering to said patient a foodstuff which has been depleted in BSA (or polypeptide of sequence SEQ ID NO: 1)

According to another aspect, the invention provides a method for depleting food, preferably child food, of BSA, in particular an amino acid sequence having a sequence SEQ ID NO: 1. Said method may be by immunoadsorption methods with immobilized antibodies.

In another aspect, the invention concerns, a method of preventing or treating a MN in a patient, preferably a child patient, which method comprises administering to said patient a functional food which has been depleted in BSA.

BSA exists in dimeric and trimeric forms under native conditions. Even if BSA is denaturated during heat treatment, it does not lose its allergenicity. BSA is also relatively resistant to enzymatic digestion. Another alternative that is proposed herein is the immunodepletion of BSA from native milk before thermal processing. In this method, immobilized antibodies specific for BSA, are covalently conjugated to polymeric beads via their Fc portion. Immunoabsorption of BSA can occur by passing milk over such beads. The collected milk will be free of BSA and can be used to production of dairy products.

The invention further provides a method of preventing or treating a Membranous Nephropathy (MN) in a child patient, which method comprises administering to said patient a foodstuff which has been depleted in Bovine Serum Albumin (BSA).

According to another aspect, the inventions concerns a method of preparing a foodstuff containing cow milk or cow milk extracts suitable for a Membranous Nephropathy (MN) child patient or for preventing onset of MN in a child patient comprising the steps consisting of depleting Bovine Serum Albumin (BSA) from said foodstuff containing cow milk or cow milk extracts, whereby a foodstuff suitable for a MN child patient or for preventing onset of MN in a child patient is prepared.

According to another aspect, the invention concerns a method for detection of food antigens, more preferably modified food antigens, using chips coated with immunoglobulin G from MN patient's sera and mass spectrometry, more preferably SELDI-TOF. In a preferred embodiment, said food antigens comprise BSA preferably cationic BSA. In an embodiment provided herein to find new modified food antigens in processed food, SELDI-TOF MS (surface-enhanced-laser-desorption ionization time-of-flight mass spectrometry) technology is used. The chip coated with IgG isolated from MN patients sera are incubated with extracts from different diets containing bovine proteins. The array is inserted into the protein chip reader that is a laser desorption ionization TOF-MS instrument. Desorbed proteins are tryptically digested and the resulting fragments identified by tandem MS.

### DEFINITIONS

A “renal disease” according to the invention refers to a disease which affects the kidney.

“Membranous nephropathy” refers to a common, form of glomerular disease and a common frequent cause of the adult idiopathic nephritic syndrome. The renal lesion is characterized by generalized diffuse granular deposits of immunoglobulin (Ig) and C3 protein in the subepithelial space which is delimited by the lamina rara external of glomerular basement membrane and the area of filtration slits. C3 protein is a complement protein of the immune system that plays a central role in the complement system and contributes to innate immunity. The patients have nephrotic syndrome and may experience an average 10% reduction in glomerular filtration rate each year.

The term “patient” or “individual” may be, for example a human or non human mammal (such as a rodent (mouse, rat), a feline, a canine or a primate) affected by or likely to be affected by renal diseases. Preferably, the subject is a human, preferably a child patient.

The term “child” or “childhood” in expressions such as “child patient” or “childhood MN” means a patient or a MN patient of less than 16 years old, more preferably patient less than 10 years old, more preferably less than 5 years old, even more preferably between 0, 3 or 5 month and 3 years old.

The term “biological sample” refers to a fluid sample isolated from an individual or from cell culture constituents, as well as samples obtained from, for example, a laboratory procedure. Biological sample refers also to one or more cells, or tissue sample from an individual such as renal sample. “Fluid samples” include, but are not limited to urine, whole blood and plasma. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

“A renal sample” of the individual refers to any material derived from the kidney of the individual, likely to contain a biological material which makes it possible to detect the expression of a gene. The renal sample is preferably a section of an individual kidney biopsy. A section of kidney biopsy to be analyzed can be obtained by any methods known in the art.

In the context of the invention, the term “treating” or “treatment” means reversing, alleviating, inhibiting the progress of, or preventing the disorder or condition to which such term applies, or one or more symptoms of such disorder or condition. In particular, the treatment of the disorder may consist in inhibiting the progress of the renal disease or renal lesions. More preferably, such treatment leads to the total eradication of the Renal disease or renal lesions.

The term “diagnosing” is used herein to refer to the identification of a molecular or pathological state, disease or condition, such as the identification of Renal disease or renal lesions or to refer to identification of a patient suffering from Renal disease or renal lesions who may benefit from a particular treatment regimen.

The term “prognosing” is used herein to refer to the prediction of the likelihood of benefit from therapy. The term “prediction” or “predicting” refers to the likelihood that a patient will respond either favourably or unfavourably to a particular therapy. In one embodiment, prediction or predicting relates to the extent of those responses. In one embodiment, the prediction or predicting relates to whether and/or the probability that a patient will lose his renal functions or renal functions will de restored following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favourably to a treatment regimen, such as a given therapeutic regimen, or whether long-term renal loss following a therapeutic regimen is likely.

BSA is meant for Bovine serum albumin (also known as “Fraction V”), a serum albumin protein having 585 amino acid residues. The full-length BSA precursor protein is 607 amino acids in length. An N-terminal 18-residue signal peptide is cut off from the precursor protein upon secretion; hence the initial protein product contains 589 amino acids. An additional 4 amino acids is cleaved to yield the mature BSA protein that contains 585 amino acids.

The term BSA refers to the protein of SEQ ID NO: 1 or the nucleic acid of sequence SEQ ID NO: 2 or their variant nucleic acid or amino acid sequences or any BSA fragment. According to the invention BSA protein contains an amino acid sequence 147 to 161 (SEQ ID NO: 3) which has been shown to be highly immunogenic. Variant nucleic acid or amino acid sequences may be naturally occurring variants sequences, such as splice variants, alleles and isoforms, or polymorphic variant thereof. Variant nucleic acid or amino acid sequences may also include respectively, nucleic acid sequences or amino acid sequences that have at least about 80% sequence identity with a nucleic acid sequence SEQ ID NO: 2 or amino acid sequence SEQ ID NO: 1, or SEQ ID NO:3. Preferably, a variant sequence will have at least about 80%, 81%, 82%, 83%, 84%, 85%, 86%, 87%, 88%, 89%, 90%, 91%, 92%, 93%, 94%, 95%, 96%, 97%, 98%, 99% nucleic acid or amino acid sequence identity to a full-length nucleic acid sequence SEQ ID NO: 2 or amino acid sequence SEQ ID NO: 1, or SEQ ID NO:3. Nucleic acid sequence identity or amino acid sequence identity is defined respectively as the percentage of nucleic acid residues or amino acid residues in the variant sequence that are identical with the nucleic acid sequence SEQ ID NO: 2 or amino acid sequence SEQ ID NO: 1, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both. Methods for sequence alignment and determination of sequence identity are well known in the art, for example using publicly available computer software such as BioPerl, BLAST, BLAST-2, CS-BLAST, FASTA, ALIGN, ALIGN-2, LALIGN, Jaligner, matcher or Megalign (DNASTAR) software and alignment algorithms such as the Needleman-Wunsch and Smith-Waterman algorithms.

Variant nucleic acid sequences include sequences capable of specifically hybridizing to the sequence of SEQ ID NO: 2 under moderate or high stringency conditions. Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/5 OmM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C.

“BSA fragment” or “polypeptide fragment” means a partially digested BSA that is a polypeptide sequence SEQ ID NO: 1 which lacks of 1 to 100 amino acids, preferably 2, 3, 4, 5, 6, 7, 8, 9, 10, 15, 20, 25, 30, 35, 40, 45, 50, 60, 70, 80, 90 amino acids, preferably, on the carboxyl terminal extremity or the amino terminal extremity. BSA may be partially, digested for example by trypsine. Preferably, the fragment contains an epitope. The smaller fragment that may be recognized by an antibody may have 4 to 5 contiguous amino acids. Consequently, according to the invention a ‘fragment’ may be of 4, 5, 6, 7, 8, 9, 10, 11, 12, 13, 14, 15, 16, 17, 18, 19, 20 contiguous amino acids. Preferably, said fragment may comprise 22 to 200 contiguous amino acids, more preferably 25 to 150 contiguous amino acids, and more preferably 30 to 100 contiguous amino acids. Preferably also, the ‘fragment’ may comprise 35 to 80 contiguous amino acids, more preferably 40 to 75 contiguous amino acids at the most, and most preferably 45 to 70 contiguous amino acids at the most.

Sequence SEQ ID NO: 4 is a BSA polypeptide fragment containing amino acids 132 to 146 of sequence SEQ ID NO:1.

The term “foodstuff” means any material, substance, additive, that can be used as food or that may be added to food.

The term “cow milk extract” means an extract of cow milk containing BSA like milk proteins or milk proteins concentrates. A cow milk extract may be a food additive containing BSA.

The term “child food” means any foodstuff designated to children, preferably, t children of less than 16 years old, more preferably less than 10 years old, more preferably less than 5 years old, even more preferably between 0, 3 or 5 months and 3 years old. For instance, child food may be Breast-milk Substitutes, baby infant formula (0-6 months) or follow-on formula (6-12 months, 12 months to 3 years) or baby meals.

The term “biomarker” means a distinctive biological or biologically-derived indicator of a process, event, or condition. According to the present invention the level of aggregation and/or the tissular localisation of the biomarker deposits according to the invention, are/is a distinctive indicator of Membranous Nephropathy, in particular, the severity of Membranous Nephropathy. The biomarker according to the invention is suitable to be used in methods of diagnosis (e.g. clinical screening), prognosis assessment; in monitoring the results of therapy, identifying patients most likely to respond to a particular therapeutic treatment, drug screening and development. Preferably, the biomarker is an antibody, a protein or peptide of BSA. The terms protein and peptide are used interchangeably herein. The biomarker may be quantified. Biomarkers and uses thereof are valuable for identification of new drug treatments and for discovery of new targets for drug treatment.

“Polypeptide” means a peptide, an oligopeptide, an oligomer or a protein comprising at least two amino acids joined together by a normal or modified peptide bond.

The term polypeptide includes short chains, known as peptides, oligopeptides and oligomers, and long chains known as proteins.

A polypeptide may be formed of amino acids modified by natural processes, such as by the post-translational maturation process or by chemical processes which are well known to the person skilled in the art. The same type of modification may be present at a plurality of locations on the polypeptide and anywhere within the polypeptide: in the peptide backbone, in the amino acid chain or even at the carboxy-terminal or amino-terminal ends. These types of modification may be the result of a natural or synthetic post-translational process, these processes being well known to the person skilled in the art.

Modification of a polypeptide means notably cationic or anionic modifications such as for example, acetylation, acylation, ADP-ribosylation, amidation, covalent binding of flavin, covalent binding of a heme, covalent binding of a nucleotide or of a nucleotide derivative, covalent binding of a lipid or of a lipid derivative, covalent binding of a phosphatidylinositol, covalent or non-covalent cross linking, cyclisation, formation of a disulphide bond, demethylation, the formation of cysteine, the formation of pyroglutamate, formylation, gamma-carboxylation, glycosylation, the formation of a GPI anchor, hydroxylation, iodisation, methylation, myristoylation, oxidation, the proteolytic process, phosphorylation, prenylation, racemisation, seneloylation, sulphation, amino acid addition such as arginylation or ubiquitination (PROTEINS STRUCTURE AND MOLECULAR PROPERTIES, 2nd Ed., T. E. Creighton, W.H. Freeman and Company, New York (1993) and Wold, F., Posttranslational Protein Modifications: Perspectives and Prospects, pgs 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663:48-62 (1992)).

Preferably BSA may be in the form of a cationic BSA. The term “cationic BSA” means a BSA having an isoelectric point (pI) above 7, preferably above 8, more preferably above 9, even more preferably between 9.5 and 10.0. Said cationic charges are preferably in the form of positively charged chemical groups such as for example by adjunction of primary amines groups or by substitution of carboxyl groups.

The isoelectric point (pI) is the pH value at which the molecule carries no electrical charge or the negative and positive charges are equal. Said pI may be measured by bi-dimensional electrophoresis or calculated by the formula pI=(pKa+pKb)/2. This formula has an even wider application since in complex ampholytes such as proteins, the isoelectric point may be approximately calculated from the equilibrium constants of the strongest acid group (pKa) and the strongest basic group (pKb) by the above equation.

“Percentage identity” between two polynucleotide or polypeptide sequences means the percentage of identical nucleotides or amino acids in the two sequences to be compared and is obtained after achieving the best alignment possible, this percentage being purely statistical and the differences between the two sequences being randomly distributed over their entire length. Comparisons between two polynucleotide or polypeptide sequences are conventionally carried out by comparing these sequences after having optimally aligned them, said comparison being carried out per segment or per “comparison window” in order to identify and compare the local regions with sequence similarity. This comparison may be carried out by means of a program, for example the EMBOSS-Needle program (Needleman-Wunsch global alignment) using the BLOSUM62 matrix/Gap opening penalty 10.0 and Gap extension penalty 0.5 (Needleman et Wunsch (1970), J. Mol. Biol. 48, 443-453 and Kruskal, J. B. (1983), An overview of sequence comparison, In D. Sankoff and J. B. Kruskal, (ed), Time warps, string edits and macromolecules: the theory and practice of sequence comparison, pp. 1-44 Addison Wesley). The term “homologous protein” or “homologous polypeptide” means a protein or a polypeptide having a percentage of identity from at least 85% with polypeptide of sequences SEQ ID NO: 1 according to the invention.

The percentage identity is calculated by determining the number of identical positions for which the nucleotide or the amino acid is identical between the two sequences, by dividing this number of identical positions by the total number of positions within the comparison window and by multiplying the result by 100.

A polynucleotide having, for example, an identity of at least 95% with the polynucleotide of SEQ ID NO: 2 is thus a polynucleotide comprising, at most, 5 modified nucleotides out of 100 nucleotides compared with said sequence. In other words, up to 5% of the nucleotides in the sequence of SEQ ID NO: 2 can be deleted or substituted by another nucleotide, or up to 5% of the total number of nucleotides in the sequence of SEQ ID NO: 2 may be inserted into said sequence. These modifications may be located at the 3′ and/or 5′ ends, or anywhere between these ends, at one or more locations.

Similarly, a polypeptide having an identity of at least 95% with the polypeptide of SEQ ID NO: 1, or SEQ ID NO: 3 is a polypeptide comprising, at most, 5 modified amino acids out of 100 amino acids compared with said sequence. In other words, by way of example, up to 5% of the amino acids in the sequence of SEQ ID NO: 1 can be deleted or substituted by another amino acid or up to 5% of the total number of amino acids in the sequence of SEQ ID NO: 1 may be inserted into said sequence. These changes to the sequence may be located at the amino-terminal and/or carboxy-terminal positions of the amino acid sequence or anywhere between these terminal positions, at one or more locations. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987).

With regard to the term “similarity”, this is calculated in the same way as identity except that amino acids which are not identical but which have common physico-chemical characteristics are considered to be identical.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., IMMUNOBIOLOGY, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) of the parent antibody, or variants thereof that retain antigen binding activity. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. The heavy chain is approximately 50 kD in size, and the light chain is approximately 25 kDa. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibodies (immunoglobulins) from any vertebrate species can be assigned to one of two clearly distinct types, called kappa (.kappa.) and lambda (.lamda.), based on the amino acid sequences of their constant domains. The ratio of the two types of light chain varies from species to species. As a way of example, the average kappa chains to lambda chains ratio is 20:1 in mice, whereas in humans it is 2:1 and in cattle it is 1:20.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An immunospecific antibody may be obtained by administering a given polypeptide to an animal followed by recovery of the antibodies produced by said animal by way of extraction from its bodily fluids. A variant of said polypeptide, or host cells expressing said polypeptide may also be administered to the animal.

An “antibody derivative” is an immune-derived moiety, i.e., a molecule that is derived from an antibody. This includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

An “antibody variant” refers herein to a molecule which differs in amino acid sequence from the amino acid sequence of a native or parent antibody that is directed to the same antigen by virtue of addition, deletion and/or substitution of one or more amino acid residue(s) in the antibody sequence and which retains at least one desired activity of the parent anti-binding antibody. Desired activities can include the ability to bind the antigen. The amino acid change(s) in an antibody variant may be within a variable region or a constant region of a light chain and/or a heavy chain, including in the Fc region, the Fab region, the CH1 domain, the CH2 domain, the CH3 domain, and the hinge region. In one embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. Ordinarily, the variant will have an amino acid sequence having at least 50% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind or the amino acid sequence of SEQ ID NO: 5 or SEQ ID NO: 6 and preferably has desired activities which are superior to those of the parent antibody. For example, the variant may have a stronger binding affinity, enhanced ability to reduce angiogenesis and/or halt tumor progression. To analyze such desired properties (for example less immunogenic, longer half-life, enhanced stability, enhanced potency), one should compare a Fab form of the variant to a Fab form of the parent antibody or a full length form of the variant to a full length form of the parent antibody, for example, since it has been found that the format of the anti-sphingolipid antibody impacts its activity in the biological activity assays disclosed herein. The variant antibody of particular interest herein can be one which displays at least about 10 fold, preferably at least about 5%, 25%, 59%, or more of at least one desired activity. The preferred variant is one that has superior biophysical properties as measured in vitro or superior activities biological as measured in vitro or in vivo when compared to the parent antibody.

An “anti-BSA agent” refers to any therapeutic agent that binds BSA (SEQ ID NO: 1 or a variant amino acid sequence) or a fragment sequence of BSA such as for example SEQ ID NO: 3, and includes antibodies, antibody variants, antibody-derived molecules or non-antibody-derived moieties that bind BSA and its variants.

An “anti-BSA antibody” or an “immune-derived moiety reactive against BSA” refers to any antibody or antibody-derived molecule that binds BSA (SEQ ID NO: 1 or a variant amino acid sequence) or a fragment sequence of BSA such as for example SEQ ID NO: 3. As will be understood from these definitions, antibodies or immune-derived moieties may be polyclonal or monoclonal and may be generated through a variety of means, and/or may be isolated from an animal, including a human subject.

A “binding partner” refers to a molecule (peptidyl or non-peptidyl) that interact directly with a target molecule such as BSA (SEQ ID NO: 1 or a variant amino acid sequence) or the amino acid sequence of SEQ ID NO: 3 and capable of neutralizing, blocking, inhibiting, abrogating, reducing or interfering with said target molecule function or metabolism. Preferably said binding partner is a protein partner or a fusion protein partner or its binding domain or an antibodies, antibody variants, antibody-derived molecules. Indeed, binding partner fusions proteins, receptor molecules and derivatives which bind specifically to said target molecule thereby sequestering its binding to one or more receptors. Said binding partner also includes antagonist variants of said target molecule, antisense molecules directed to said target molecule, RNA aptamers, and ribozymes against said the target molecule.

The term “immunospecific” applied to the term antibody, in relation to a given polypeptide, means that the antibody has a greater affinity for this polypeptide than for other polypeptides known from the prior art. Said polypeptide may be according to the invention a sequence SEQ ID NO: 1 or of the invention, a variant thereof or a cationic BSA or BSA deposits.

“Positive” biological sample means a serum containing antibodies produced following an immunogenic reaction with a polypeptide having a sequence SEQ ID NO: 1 or SEQ ID NO: 5 of the invention, a variant thereof or a cationic BSA or BSA deposits. A positive biological sample may also be a biological sample containing cationic BSA or cationic BSA fragments or BSA deposits.

“BSA deposits” means complexes found in biological samples containing at least protein aggregates and immunoglobulins such as IgG4 and/or IgG1. Those deposits may further contain complement proteins such as C3 proteins or C5b-9 proteins complexes “Antigen” means any compound which, either alone or in combination with an adjuvant or carrier, is capable of inducing a specific immune response. This definition also includes any compound exhibiting structural similarity with said antigen capable of inducing an immunological response directed against said antigen.

An “epitope” or “antigenic determinant” refers to that portion of an antigen that reacts with an antibody antigen-binding portion derived from an antibody.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

Techniques for detecting antibody binding are well known in the art. Antibody binding to a protein of interest may be detected through the use of chemical reagents that generate a detectable signal. In one method, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Samples may be examined via automated microscopy or by personnel with the assistance of computer software that facilitates the identification of positive staining cells.

### FIGURES

FIG. 1. Serum anti-BSA antibodies and epitope mapping

This figure shows absorbance values of a specific ELISA to assay antibodies to BSA in sera from patients with membranous nephropathy (MN), from patients with other types of glomerular diseases (DC, disease control) or from healthy controls (C), divided into three groups according to age: <5yrs; 5-16 yrs; and adult. Nonparametric statistical tests (Mann-Whitney U test) with Bonferroni correction showed a statistical difference between MN and both DC and C sera in children <5 yrs. In the second group of children a statistical difference was observed only between MN and DC. Individual patients are represented by circles, and medians for each group by horizontal lines. A value of p<0.008 was considered statistically significant. *p<0.008.

FIG. 2. Detection and characterization of circulating BSA

This figure shows the amount of circulating BSA assessed by ELISA in sera from patients with membranous nephropathy (MN), from patients with other types of glomerular diseases (DC), or from controls (C). All patients and controls were divided into three groups according to the age: <5 yrs; 5-16yrs; and adult. Nonparametric statistical tests (Mann-Whitney U test) with Bonferroni correction showed a statistical difference between MN and both DC and C sera in youngest children and adult age groups. Individual patients are represented by circles, and medians for each group by horizontal lines. A value of p<0.008 was considered statistically significant.*p<0.008.

FIG. 3. Amount, circulating BSA and disease activity in a patient with membranous nephropathy

Serum samples were collected from patient #4. The patient is a 2-year, 3-month-old Caucasian male who was diagnosed in June 2004 with membranous nephropathy (stage I) responsible for severe nephrotic syndrome (proteinuria, 9.45 g/24 h; albuminemia, 21.8 g/; serum creatinine, 30 pmol/l). He was treated for 4.5 months with prednisone started at a dose of 60 mg/m2/day for one month, tapered to 60 mg/m2 every other day for 2 months and progressively stopped over a period of 6 weeks, with resolution of proteinuria within 3 months. At the age of 4 years, 6 months (November 2006), a relapse of initial disease occurred (proteinuria 10.1 g/24 h; albuminemia, 25.6 g/l; serum creatinine 18 pmol/l). Kidney biopsy showed stage I-II membranous nephropathy. He was treated with the same prednisone treatment protocol as initially, with complete normalization of all serum parameters and disappearance of proteinuria. In December 2008, proteinuria was in the normal range with normal renal function. Complete remission persisted at last follow-up at the age of 8 years.

FIG. 4. Principle of the test strip:

A membrane coated with several purified antigens is incubated with a serum sample to be test. If the sample tested is positive, specific antibodies in the serum sample bind specifically to said coupled antigens (see FIG. 4A). Antibodies from the serum tested which are bind to the antigens, are recognized by an alkaline phosphatase-labelled anti-human antibodies.

FIG. 5. Schematic principles of peptide based ELISA

FIG. 6. Method for determining isoelectric point of circulating BSA

### DETAILED DESCRIPTION OF THE INVENTION

The inventors have described a distinct form of membranous nephropathy in children aged 5 months to 2.3 years whose presentations were otherwise typical of idiopathic membranous nephropathy. The inventors have demonstrated that these patients have both high-level anti-BSA antibodies of IgG1 and IgG4 subclasses and circulating cationic BSA. Indeed, BSA has been colocalized with IgG in subepithelial immune deposits. IgG1 and IgG4 eluted from kidney-biopsy specimen had anti-BSA reactivity. These data strongly suggest that in these patients, cationic BSA became planted into the anionic glomerular capillary wall, which led to the subsequent deposition of anti-BSA IgG.

Although circulating antibodies to BSA have been detected in many human sera, they were not associated with any detectable clinical event (Mogues T. 2005 J Immunol Methods; 300:1-11.), except for IgE-mediated cow's milk allergy (Fiocchi A. 1995 J Am Coll Nutr; 14:239-44). The inventors provide the evidence that in patients with membranous nephropathy, most anti-BSA antibodies were directed against a peptide of BSA that comprises amino-acid residues 147 to 161.

The present invention arises from the unexpected discovery, by the inventors, that BSA deposits and anti-BSA antibodies are found only in childhood MN. This phenomenon seems to be linked with childhood metabolism. Indeed, heat treatment of BSA denatures the protein and results in reduced proteolysis, (Alting A C, 1997 Diabetes Care; 20:875-80.) in the relatively high pH (3-4) of the infants' stomach compared with that of adults (pH2) (Schmidt D G, 1995 Clin Exp Allergy.; 25:1007-17). Furthermore, the amount of intact BSA entering the circulation is likely higher during infancy before the gastrointestinal tract has matured and its barrier function has been established (Van Elburg R M. 2003 Arch Dis Child Fetal Neonatal Ed.; 88:F52-5; Sreedharan R, 2004 Gastrointestinal tract. Pediatrics; 113:1044-50). This amount may be increased during childhood gastroenteritis (Torente F. 2004 Pediatric gastrointestinal disease. 4th ed. Hamilton, ON, Canada: BC Decker; p. 944-958). Both the predominance of a cationic form and the increased amount of absorbed BSA most likely contribute to the development of membranous nephropathy in young children.

The inventors equally provide that the patients with BSA-induced membranous nephropathy produced mainly IgG4 accompanied by IgG1 antibodies. IgG4 is unique among the IgG subclasses because it weakly activates complement, and behaves mainly as a monovalent Ig (van der Zee J S. 1986 J Immunol; 137:3566-71; van der Zee J S. 1986 Clin Exp Immunol; 64:415-22; Aalberse R C, Schuurman J. 2002 Immunology; 105:9-19), Therefore, IgG4 can form small-sized, non precipitating immune complexes that escape clearance and are poorly detected. Although direct interaction of cationic BSA with anionic glomerular capillary wall most likely is the triggering event, one can speculate that circulating IgG4-containing immune complexes may subsequently be involved because of their longer half-life and their possible dissociation at the glomerular endothelium site, owing to the usual low affinity of IgG4 for antigens (Oliveira D B. 1998 Lancet; 351:670-1).

### EXAMPLE

Patients

The inventors analyzed a consecutive cohort of 9 children and 41 adults with idiopathic membranous nephropathy all biopsied between 2004 and 2009. These patients lacked features of secondary membranous nephropathy. Their clinical characteristics are presented in the Supplementary

No manifestation of cow's milk allergy was observed in any of the patients. Serum specimens were also obtained from age-matched patients with other glomerular diseases (N=63) and nonproteinuric (N=109). A Committee for Persons Protection approved the study, and written informed consent was obtained from all adults, parents of children under age 10 years, and assent from children over age 10 with consent of their parents.

BSA IGG Antibodies and Circulating Immune Complexes

Circulating antibodies were detected on ELISA plates coated with BSA (Sigma). Circulating immune complexes containing C1q, or C3d, were detected by using EIA kits (Quidel Co.)

Peptide Based Elisa Assay

A panel of selected peptides was purchased from Mimotopes (Mimotopes. The Peptide Company). The peptide solutions were covalently immobilized in the wells, and 100-fold diluted sera were applied for assays. IgG antibodies to peptides were detected using alkaline phosphatase conjugated anti-human IgG (Sigma)

Circulating BSA

BSA was detected in patients' sera with the Bovine Albumin ELISA kit (Alpha Diagnostic Intl.). BSA was immunopurified from patient's and controls' sera or bovine serum by affinity chromatography using anti-bovine albumin agarose (Sigma). Immunopurified BSA was also analyzed by two-dimensional electrophoresis. The first dimension was run on IPG ready strips

pH 3-10 (BioRad), and the second on 8% SDS-PAGE. The separated proteins were blotted on PVDF membrane and the position of BSA was determined with chicken HRP conjugated anti-BSA antibodies (GeneTex, Inc).

Western Blotting and Elution of IG

BSA and human serum albumin (HSA) were electrophoresed and transferred to PVDF membranes, according to standard protocols. Detection antibodies were peroxidase-conjugated goat anti-human antibody (Chemicon). IgG subclasses were identified by mouse monoclonal anti-human IgG1, IgG2, IgG3, and IgG4 antibodies, respectively (Margaret Goodall, Birmingham University, UK), followed by peroxidase-conjugated sheep anti-mouse IgG (GE Healthcare). Immunoglobulins were acid-eluted from the cores of kidney biopsy specimens from patients with membranous nephropathy. The eluted IgG was used to immunoblot the BSA and HAS directly.

Immunohistologic Analysis

The inventors analyzed cryosections or paraffin embedded sections of normal human kidney and biopsy specimens from the patients with membranous nephropathy and with other glomerular diseases.

The inventors detected BSA in cryosections with a rabbit polyclonal anti-BSA antibody (Invitrogen,) and PLA2R in paraffin embedded sections with a rabbit polyclonal anti-PLA2R antibody (Atlas antibodies AB,) followed by goat Alexa 488-conjugated anti-rabbit Fab IgG antibody (Molecular Probes). Colocalization of BSA and IgG was analyzed by confocal microscopy. Cryosections of the biopsy specimen were first incubated with rabbit polyclonal anti-BSA antibodies (Invitrogen), then with goat Alexa 488-conjugated anti-rabbit Fab IgG antibodies and goat Alexa 568-conjugated anti-human IgG (Molecular probes). The cryosections were also stained with mouse monoclonal anti-human IgG1, IgG2, IgG3, and IgG4 antibodies followed by rabbit Alexa 488 conjugated anti-mouse antibodies. Sections were examined under a confocal microscope Leica TCS-SP2 and analyzed with Leica Confocal Software version 2.61.

Statistical Analysis

The nonparametric Mann-Whitney U test with Bonferroni correction was used for comparison of anti-BSA antibodies or circulating BSA level in patients with MN as compared to control subjects in each age group. Values of p<0.008 were considered statistically significant.

Results

Serum Anti-BSA Antibodies and Epitope Mapping

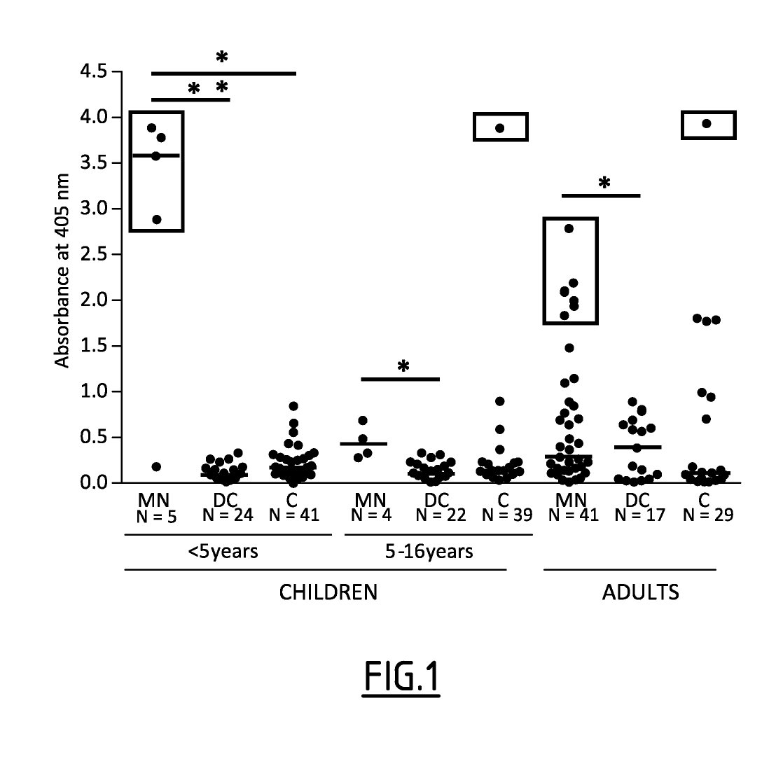

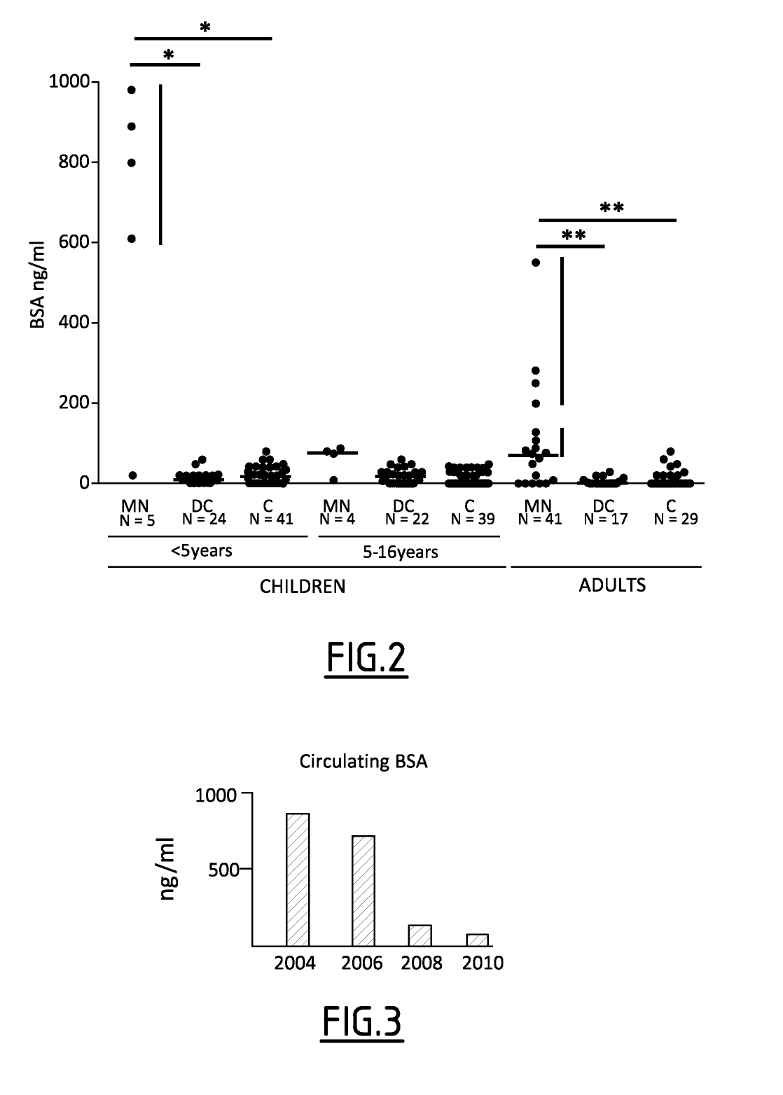

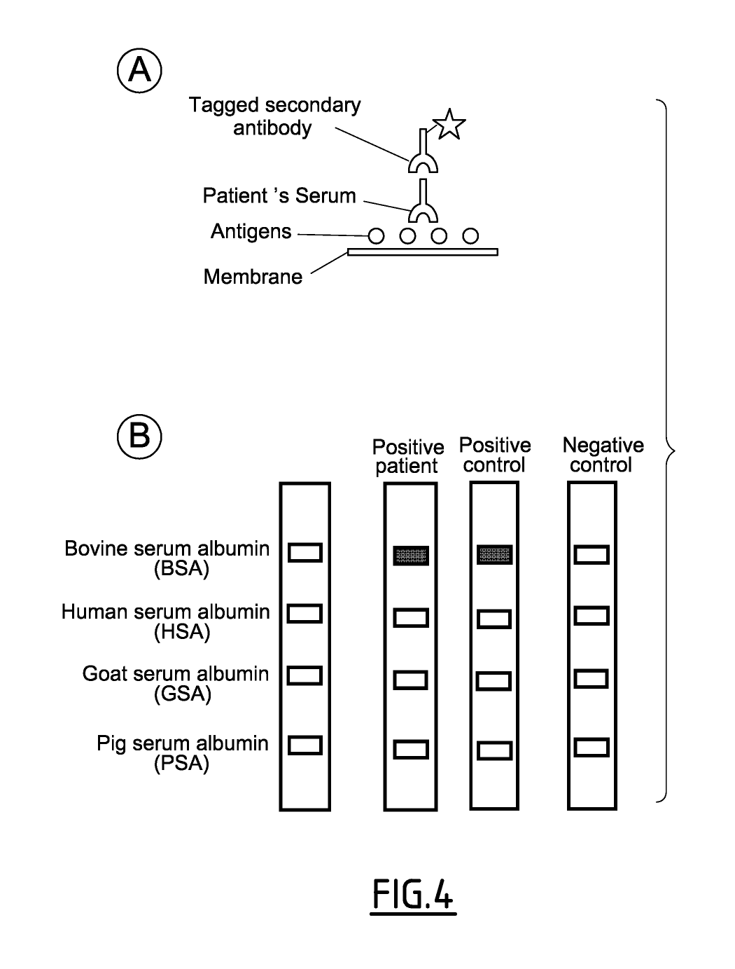

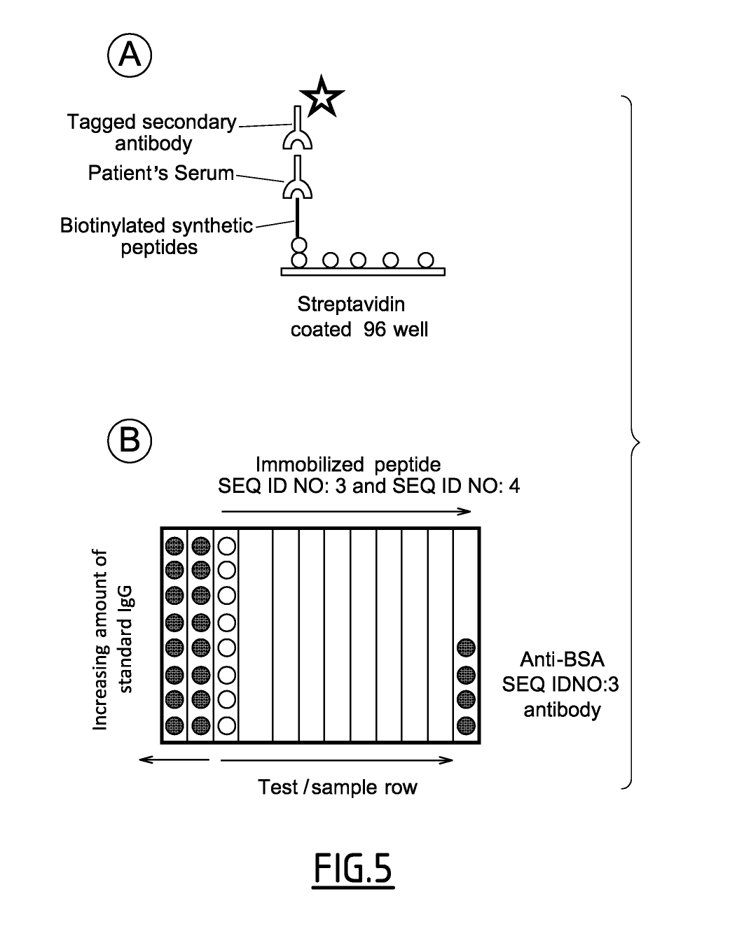

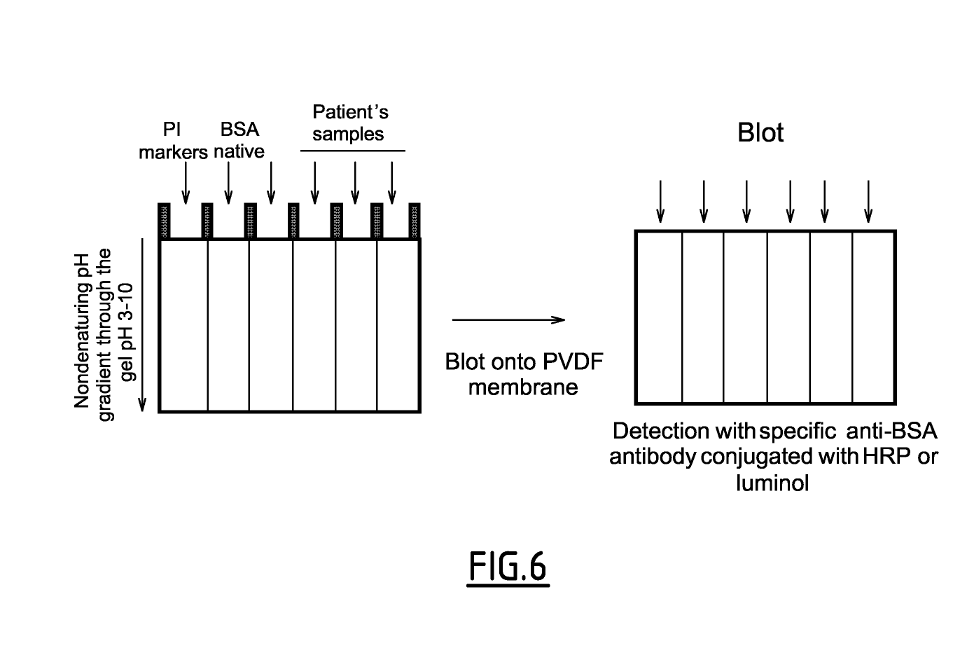

Because anti-BSA antibodies are common in the general population, the inventors asked whether they could be related to the pathogenesis of membranous nephropathy and recognize specific epitopes. High levels of anti-BSA antibodies were found in 4 of 5 consecutive children in the first age group (<5 yrs) and 7 of 41 consecutive adults with membranous nephropathy (FIG. 1). Disease controls had lower levels of anti-BSA antibodies in both child groups. Nonproteinuric controls had lower levels of anti-BSA antibodies in the age group <5 years. Two controls in the older age groups had high levels of anti-BSA antibodies. Serum samples from the patients and controls with the highest levels of anti-BSA antibody by ELISA recognized BSA but not HSA by Western blotting. Anti-BSA antibodies were mainly of IgG1 and IgG4 subclasses with either predominance of IgG4 or IgG1. In contrast to the patients with IgE-mediated BSA allergy, the inventors did not detect increased level of anti-BSA IgE (not shown).

The inventors hypothesized that BSA specific antibodies reacted primarily with sequential epitopes in which the amino acid sequences differ greatly between BSA and HSA. Fourteen peptide candidate epitopes corresponding to dissimilarity regions were synthesized. All BSA responses in patients with membranous nephropathy predominantly targeted the BSA peptide 147-161 while controls with the higher level of anti-BSA antibodies showed a broader spectrum of reactivity toward the synthesized peptides. Peptide 147-161 contains two linear epitopes that are not present in HSA.

Identification and Characterization of Circulating BSA

Because trypsin specifically cleaves proteins on the carboxyl side of Arg and Lys residues, the BSA peptide 147-161 should be broken down in the gut. Therefore, the inventors speculated that in pathologic conditions, a substantial amount of the BSA protein was not, or was only partially, digested. The 4 children with high-level anti-BSA antibodies also had high levels of circulating BSA (FIG. 2). Among the 7 adults with membranous nephropathy and high-level anti-BSA antibodies, 4 also had elevated levels of circulating BSA albeit in lower range (FIG. 2). The two controls with high-level of anti-BSA antibodies had very low level of circulating BSA.

The apparent molecular weight of the BSA reactive antigen was assessed by SDS-PAGE after immunopurification from patients' sera. BSA antigen migrated slightly faster than the native BSA immunopurified in the same conditions. Positively charged, cationic proteins can attach to the glomerular basement membrane and serve as target for in situ immune complex formation. The cationic form of BSA, but not the native form which is slightly anionic, induced membranous nephropathy in various animal models. Therefore, the inventors analyzed by 2D SDS-PAGE, the BSA immunopurified from patients' sera. BSA circulating in children with membranous nephropathy migrated in the basic range of pH whereas native BSA migrated in neutral or slightly acidic regions. In contrast, in adult patients, immunopurified BSA migrated as native BSA or was below detection limits. Three to six spots of cationic BSA (cBSA) circulating in sera of children with membranous nephropathy were observed by 2D SDS-PAGE. Each spot is reactive to anti-BSA antibody and migrates at the same apparent molecular weight than native BSA. Said spots are cationic BSA isovariants having a pI between 8 and 9.5, meaning that, three to five cationic BSA isovariants may be detected in children plasma.

Said cationic BSA isovariants are not observed in adult extract nor in purified BSA. Consequently, those isovariants are specific of children having membranous nephropathy.

Despite the presence of high-level circulating BSA and anti-BSA antibodies, the inventors failed to detect significant amounts of complement-binding circulating immune complexes by 2 different assays (CIC-C1q EIA less than 4 μg Eq/ml; CIC-Raji EIA less than 10 μg Eq/ml.).

Colocalization of BSA and IGG in Immune Deposits and Anti-BSA Activity of Eluted IG

The inventors searched for BSA deposits in glomeruli in the 7 patients with high-level anti-BSA antibodies and circulating BSA, and in 15 additional patients with idiopathic membranous nephropathy but without circulating BSA irrespective of anti-BSA antibodies. Subepithelial granular deposits of BSA were visible only in children who had both high level circulating cationic BSA and BSA specific antibodies. No staining was seen in biopsy specimens from patients with idiopathic membranous nephropathy but without circulating cationic BSA irrespective of the presence or the absence of anti-BSA antibodies. In patients with positive staining for BSA, there was no M-type PLA2R in immune deposits.

However, PLA2R was detected in 14 of the 20 biopsies without BSA deposits, while the 6 remaining ones negative for BSA were also negative for PLA2R.

Normal kidney tissue or biopsy specimen from patients with other nephropathies stained negative for BSA. These overall results strongly suggest that children #1 to 4 developed cBSA-related membranous nephropathy.

BSA and IgG were colocalized in many areas of the outer aspect of the capillary wall in a fine granular pattern. Quantitative analysis of the fluorescence showed a complete superimposition of the two. Specificity of BSA staining was assessed in double-labeling confocal studies by preincubating anti-BSA antibodies with pure BSA and by using anti-HSA instead of anti-BSA antibodies. Biopsy specimen also showed abundant subepithelial deposits of the membrane attack complex C5b-9.

The inventors eluted Ig from biopsy specimen of one patient having BSA glomerular deposits and of 4 patients with membranous nephropathy but without BSA deposits. Reactivity of IgG was analyzed by Western blotting with BSA or HSA. Results shown in Fig H demonstrated the presence of anti-BSA IgG4 and IgG1 only in the patient with BSA deposits, while no reactivity was found with HSA. These results are in keeping with the predominance of IgG1 and IgG4 subclasses in subepithelial deposits a characteristic feature of membranous nephropathy, and with the Ig subclass reactivity profile observed in serum.

Association with Disease Activity

All four children with cBSA-related membranous nephropathy underwent a complete or partial remission. The inventors analyzed serum specimens collected serially from one child. The inventors found high level of anti-BSA IgG4 and IgG1 antibodies and circulating BSA (FIG. 3) when there was clinically significant disease activity, as measured by urinary protein. During remission, there was a substantial decrease of circulating BSA and anti-BSA antibodies.

1. A method for diagnosing or prognosing Membranous Nephropathy (MN) in a child patient, which method comprises detection of
(i) cationic Bovine Serum Albumin (BSA) or a cationic polypeptide comprising sequence SEQ ID NO: 3 and/or
(ii) an antibody that binds to a polypeptide of sequence SEQ ID NO: 3, in a biological sample from said child patient.

2. The method according to claim 1, wherein said child patient is less than 10 years old.

3. The method according to claim 1, herein said cationic polypeptide comprises the amino acid sequence of SEQ ID NO: 1 or a fragment thereof.

4. The method according to claim 1, wherein said cationic BSA or cationic polypeptide has an isoelectric point (pI) above 7.5.

5. The method according to claim 1, wherein said antibody is an IgG1 and/or IgG4.

6-8. (canceled)

9. The method according to claim 1, wherein said cationic BSA and/or cationic polypeptide has a pI between 9.5 and 10.0.

10. An isolated antibody or antibody fragment which binds specifically to an amino acid sequence SEQ ID NO: 3.

11. A kit for detecting antibodies directed against a polypeptide of sequence SEQ ID NO: 3 in a biological sample of an individual comprising:
(i) a polypeptide comprising SEQ ID NO: 3; and/or,
(ii) a polypeptide comprising sequence SEQ ID NO: 1 and/or,
(iii) an homologous polypeptide comprising or consisting of a sequence having at least 90% identity with sequence SEQ ID NO: 1; and/or,
(iv) a fragment of said polypeptide defined in (ii) and/or fragment of said homologous polypeptide sequence defined in (iii) provided said fragment or homologous polypeptide binds an antibody which binds specifically to an amino acid sequence SEQ ID NO: 3, and optionally,
(v) an antibody or antibody fragment which binds specifically to an amino acid sequence SEQ ID NO: 3 or a composition comprising such an antibody or antibody fragment and/or,
(vi) a polypeptide of sequence SEQ ID NO: 4.

12. A kit for detection of cationic BSA or cationic polypeptide comprising sequence SEQ ID NO: 3 in a biological sample of an individual comprising:
a) means for capturing circulating BSA
b) an antibody or antibody fragment which binds specifically to an amino acid sequence SEQ ID NO: 3 or a composition comprising such an antibody or antibody fragment and/or
c) means for determining the isoelectric point of a polypeptide.

13. A foodstuff likely to contain Bovine Serum Albumin (BSA) or cow milk or cow milk extracts wherein said foodstuff is depleted in BSA.

14. The foodstuff according to claim 13 wherein said foodstuff is a dairy food product.

15. The foodstuff according to claim 13 wherein said foodstuff contains powdered milk or condensed milk.

16. The foodstuff according to claim 13 wherein said foodstuff is a child food.

17. A method of preventing or treating a Membranous Nephropathy (MN) in a child patient, which method comprises administering to said patient a foodstuff which has been depleted in Bovine Serum Albumin (BSA).

18. A method of preparing a foodstuff containing cow milk or cow milk extracts suitable for a Membranous Nephropathy (MN) child patient or for preventing onset of MN in a child patient, wherein said method comprises depleting Bovine Serum Albumin (BSA) in said foodstuff.

19. A method for detecting food antigens, said method comprising incubating chips coated with immunoglobulin G from MN patient's sera with food extracts containing bovine proteins and identifying said food antigens by mass spectrometry.

20. The method according to claim 16, wherein said food antigens are modified food antigens.

21. The method according to claim 16, wherein mass spectrometry is SELDI-TOF.

22. A method of preventing or treating a Membranous Nephropathy (MN) in a child patient, wherein said method comprises removing from said patient antibodies which bind to a polypeptide of sequence SEQ ID NO: 1.

23. The method according to claim 19, wherein said antibodies specifically bind to a polypeptide of sequence SEQ ID NO: 3.

24. A method of preventing or treating a Membranous Nephropathy (MN) in a child patient, wherein said method comprises:
a) detecting (i) cationic Bovine Serum Albumin (BSA) or a cationic polypeptide comprising sequence SEQ ID NO: 3 and/or (ii) an antibody that binds to a polypeptide of sequence SEQ ID NO: 3, in a biological sample from said child patient,
b) when deducing that said child patient is affected by Membranous Nephropathy, administering to said patient a therapeutic agent or a foodstuff which has been depleted in Bovine Serum Albumin (BSA), or removing from said patient antibodies which bind to a polypeptide of sequence SEQ ID NO: 1.
